(19) United States
(12) Patent Application Publication
(10) Pub. No.: US 2005/0267124 A1
(43) Pub. Date: Dec. 1, 2005

(54) Pharmaceutical compositions comprising NEP-inhibitors, inhibitors of the endogenous producing system and PDEV inhibiitors

(75) Inventors: Dieter Ziegler, Hemmingen (DE); Klaus Witte, Hannover (DE); Matthias Straub, Barsinghausen (DE); Michael Weske, Burgdorf (DE); Dagmar Hoeltje, Gehrden (DE)

Correspondence Address: CROWELL & MORING LLP;INTELLECTUAL PROPERTY GROUP, P.O. BOX 14300, WASHINGTON, DC, 20044-4300, US

(73) Assignee: Solvay Pharmaceuticals GmbH, Hannover (DE)

(21) Appl. No.: 11/127,163

(22) Filed: May 12, 2005

Related U.S. Application Data
(60) Provisional application No. 60/570,829, filed on May 14, 2004.

(51) Int. Cl. A61K 31/519

(52) U.S. Cl. 514/252.16

(57) ABSTRACT

A combination therapy for the inhibition and/or treatment of sexual dysfunction and/or cardiovascular diseases in humans and other mammals, by administering a synergistic combination of at least one inhibitor of neutral endopeptidase, at least one inhibitor of the endogenous endothelin producing system, and at least one inhibitor of phosphodiesterase 5.

### CROSS REFERENCE TO RELATED APPLICATIONS

This application claims priority from U.S. provisional patent application No. 60/570,829, filed May 14, 2004, the entire disclosure of which is incorporated herein by reference.

### BACKGROUND OF THE INVENTION

The present invention relates to a novel combination therapy for the treament or inhibition of sexual dysfunction, endothelial dysfunction and/or cardiovascular diseases, by a synergistic combination of at least one inhibitor of neutral endopeptidase (=NEP), at least one inhibitor of the endogenous endothelin producing system and at least one inhibitor of phosphodiesterase 5 (=PDE V inhibitor). Thus, the invention also relates to novel pharmaceutical compositions comprising NEP inhibitors, inhibitors of the endogenous endothelin producing system and PDE V inhibitors and the use of said pharmaceutical composition in the inhibition or treatment of cardiovascular diseases in mammals and humans.

The nature of cardiovascular, in particular hypertensive vascular, diseases is multifactorial. Combination therapy has been shown to address the multiple pathophysiologic factors that play a role in blood pressure elevation, including blood volume, vasoconstriction, and the impact of sympathetic nervous system and Renin-Angiotensin-Aldosterone-System (=RMS) activity (see e.g. M. R. Weir, American Journal of Hypertension 11 (1998) 163S-169S), potentially resulting in both greater reduction in blood pressure and in lowered risks for target-organ damage. The use of a fixed, low-dose combination agent could also offer lower doses of each component than those that may be necessary with monotherapy, thus reducing the risks of dose-dependent adverse events and associated compliance problems.

Sexual dysfunction (SD) is a significant clinical problem which can affect both males and females. The causes of SD may be both organic as well as psychological. Organic aspects of SD are typically caused by underlying vascular diseases, such as those associated with hypertension or diabetes mellitus, by prescription medication and/or by psychiatric disease such as depression. Physiological factors include fear, performance anxiety and interpersonal conflict. SD impairs sexual performance, diminishes selfesteem and disrupts personal relationships thereby inducing personal distress.

From document WO 99/55726 A1 it is known, that certain thiol inhibitors of ECE are useful for treating or inhibiting i.a. erectile dysfunction.

Document EP 1 097 719 A1 discloses the use of NEP inhibitors for the treatment of FSD.

In document WO 02/03995 A2, the use of NEP inhibitors or of combinations of NEP inhibitors with PDE5 inhibitors is described.

Publication WO 02/06492 A1 discloses i.a. antibodies against and inhibitors of a specific polypeptide having soluble secreted endopeptidase (=SEP) activity.

Compounds with a dually acting combined inhibitory effect on NEP and the endogenous endothelin producing system, i.e. benzazepine-, benzoxazepine- and benzothiazepine-N-acetic acid derivatives, are known from document EP 0 733 642 A1 (=U.S. Pat. No. 5,677,297). Further favourable pharmacological properties of compounds falling within the structural scope of EP 0 733 642 A1 are known from documents EP 0 830 863 A1 (=U.S. Pat. No. 5,783,573), WO 00/48601 A1 (=U.S. Pat. No. 6,482,820) and WO 01/03699 A1 (=US-2003-0040512-A1).

Phosphonic acid substituted benzazepinone-N-acidic acid derivatives with a combined inhibitory effect on NEP and the endogenous endothelin producing system are disclosed in document EP 0 916 679 A1 (=U.S. Pat. No. 5,952,327).

Amidomethyl-substituted 1-(carboxyalkyl)-cyclopentylcarbonylamino-benzazepine-N-acetic acid derivatives which are useful e.g. for the inhibition and/or treatment of cardiovascular conditions or diseases, are disclosed in document WO 2005/030795 A1.

From document WO 02/094176 A2 it is known that certain compounds, including those disclosed in document EP 0 733 642 A1 and in document EP 0 916 679 A1, may inhibit the endogenous endothelin producing system via an inhibition of metalloprotease IGS5. The metalloprotease IGS5 is also known as human soluble endopeptidase (=hSEP) and is described e.g. in document WO 02/094176 A2. Further, WO 02/094176 A2 discloses the use of compounds with combined NEP/hSEP inhibitory activity for the inhibition or treatment of inter alia cardiovascular diseases.

### SUMMARY OF THE INVENTION

It is the object of the present invention to provide a novel combination therapy for sexual dysfunction, endothelial dysfunction and/or cardiovascular diseases, with enhanced efficacy and a favorable safety profile.

It has now surprisingly been found that a combination of at least one NEP-inhibitor, at least one inhibitor of the endogenous endothelin producing system and additionally at least one PDE V inhibitor, provides still further enhanced efficacy in sexual dysfunction, endothelial dysfunction and/or cardiovascular diseases, and a favourable safety profile.

The invention therefore relates in a first aspect to pharmaceutical compositions comprising pharmacologically effective quantities of each of
a) at least one NEP-inhibitor as a first active agent,
b) at least one inhibitor of the endogenous endothelin producing system as a second active agent and
c) at least one PDE V inhibitor as a third active agent.

The pharmaceutical compositions according to the invention may further and preferably comprise conventional pharmaceutically acceptable auxiliaries and/or carriers. The pharmaceutical compositions according to the invention may further comprise acetylsalicylic acid.

Inhibitors of the endogenous endothelin producing system can be selected from the group consisting of inhibitors of ECE, inhibitors of hSEP and dually acting compounds capable of inhibiting ECE and hSEP. Dually acting compounds capable of inhibiting ECE and hSEP are preferred.

In the pharmaceutical compositions according to the invention, the subcombination of at least one NEP-inhibitor (a) and at least one inhibitor of the endogenous endothelin producing system (b) can preferably be realized by a dually acting compound capable of inhibiting NEP and the endogenous endothelin producing system. Preferred are dually acting compounds capable of inhibiting NEP and hSEP. Particularly preferred are the dually acting compounds corresponding to Formula I, 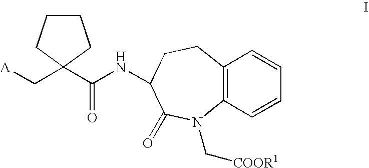
wherein
R¹ is hydrogen or a group forming a biolabile carboxylic acid ester
A represents a group selected from the subgroups a, 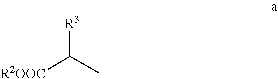
   wherein
R² is hydrogen or a a group forming a biolabile carboxylic acid ester and
R³ is a phenyl-C₁₋₄-alkyl group which can optionally be substituted in the phenyl ring by C₁₋₄-alkyl, C₁₋₄-alkoxy or halogen; or a naphthyl-C₁₋₄-alkyl group; or
b, 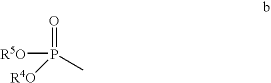
   wherein
R⁴ is hydrogen or a group forming a biolabile phosphonic acid ester and
R⁵ is hydrogen or a group forming a biolabile phosphonic acid ester; or c, 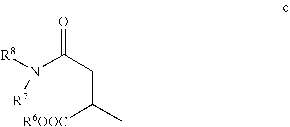
   wherein
R⁶ is is hydrogen or a group forming a biolabile carboxylic acid ester,
R⁷ is hydrogen, C₁₋₄-alkyl or C₁₋₄-hydroxyalkyl, the hydroxyl group of which is optionally esterified with C₂₋₄-alkanoyl or an amino acid residue, and
R⁸ is C₁₋₄-alkyl; C₁₋₄-alkoxy-C₁₋₄-alkyl; C₁₋₄-hydroxyalkyl, which is optionally substituted by a second hydroxyl group and the hydroxyl groups of which are each optionally esterified with C₂₋₄-alkanoyl or an amino acid residue; (C₀₋₄-alkyl)₂amino-C₁₋₆-alkyl; C₃₋₇-cycloalkyl; C₃₋₇-cycloalkyl-C₁₋₄-alkyl; phenyl-C₁₋₄-alkyl, the phenyl group of which is optionally substituted 1-2 times by C₁₋₄-alkyl, C₁₋₄-alkoxy and/or halogen; naphthyl-C₁₋₄-alkyl; C₃₋₆-oxoalkyl; phenylcarbonylmethyl, the phenyl group of which is optionally substituted 1-2 times by C₁₋₄-alkyl, C₁₋₄-alkoxy and/or halogen, or 2-oxoazepanyl, or
R⁷ and R⁸ together are C₄₋₇-alkylene, the methylene groups of which are optionally replaced 1-2 times by carbonyl, nitrogen, oxygen and/or sulfur and which are optionally substituted once by hydroxy, which is optionally esterified with C₂₋₄-alkanoyl or an amino acid residue; C₁₋₄-alkyl; C₁₋₄-hydroxyalkyl, the hydroxyl group of which is optionally esterified with C₂₋₄-alkanoyl or an amino acid residue; phenyl or benzyl,
   and/or physiologically compatible salts of acids of Formula I and/or physiologically compatible acid addition salts of compounds of Formula Ic.

Where the substituents in the compounds of Formula I are or contain C₁₋₄-alkyl groups, these may be straight-chain or branched. Where biolabile ester forming groups in the compounds of Formula I are or contain lower alkyl groups, these may be straight-chain or branched and contain usually 1 to 4 carbon atoms. Where the substituents contain halogen, fluorine, chlorine or bromine, preferably fluorine or chlorine, are particularly suitable. Where substituents contain C₂₋₄-alkanoyl, this may be straight-chain or branched. Acetyl is preferred as C₂₋₄-alkanoyl.

Where substituents are biolabile ester forming groups, these as a rule represent prodrugs of the active drug prinicple. Prodrugs are therapeutic agents which are inactive per se but are transformed into one or more active metabolites. Prodrugs are bioreversible derivatives of drug molecules used to overcome some barriers to the utility of the parent drug molecule. These barriers include, but are not limited to, solubility, permeability, stability, presystemic metabolism and targeting limitations (see e.g. Medicinal Chemistry: Principles and Practice, 1994, ISBN 0-85186-494-5, Ed.: F. D. King, p. 215; J. Stella, “Prodrugs as therapeutics”, Expert Opin. Ther. Patents, 14(3), 277-280, 2004; P. Ettmayer et al., “Lessons learned from marketed and investigational prodrugs”, J.Med.Chem., 47, 2393-2404, 2004).

Suitable physiologically compatible salts of free acids or partial esters of Formula I include their alkali metal, alkaline earth metal or ammonium salts, for example sodium or calcium salts or salts with physiologically compatible, pharmacologically neutral organic amines such as, for example, diethylamine or tert.-butylamine.

Preferred are the compounds corresponding to Formula Ia, 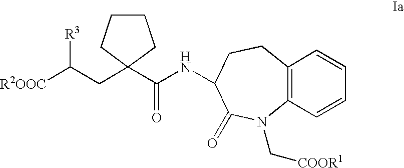
wherein R¹, R² and R³ have the above meanings, and physiologically compatible salts of acids of Formula la. Preferred salts of compounds of Formula la are e.g. disclosed in document WO 03/059939 A1 which is incorporated herein by reference. The compounds of Formula la contain two chiral carbon atoms, namely the carbon atom which is in the 3 position of the ring framework (=3-position) and bears the amide side-chain, and the carbon atom of the amide side-chain which bears the radical R³ (=2′-position). The compounds can therefore exist in several optically active stereoisomeric forms or as a racemate. According to the present invention both the racemic mixtures and the isomerically pure compounds of Formula la may be used.

The compounds of Formula Ia are optionally esterified dicarboxylic acid derivatives. Depending on the form of administration, biolabile monoesters, particularly compounds in which R² is a group forming a biolabile ester and R¹ is hydrogen, or dicarboxylic acids are preferred, the latter being particularly suitable for i.v. administration. Groups which can be cleaved under physiological conditions in vivo, releasing bioavailable derivatives of the compounds of Formula la, are suitable as groups forming biolabile carboxylic acid esters R¹ and R². Suitable examples of this are Cl4-alkyl groups, in particular methyl, ethyl, n-propyl and isopropyl; C₁₋₄-alkyloxy-C₁₋₄-alkyloxy-C₁₋₄-alkyl groups, in particular methoxyethoxymethyl; C₃₋₇cycloalkyl groups, in particular cyclohexyl; C₃₋₇-cycloalkyl-C₁₋₄-alkyl groups, in particular cyclopropylmethyl; N,N-di-(C₀₋₄-alkyl)amino-C₁₋₆-alkyl groups; phenyl or phenyl-C₁₋₄-alkyl groups optionally substituted in the phenyl ring once or twice by halogen, C₁₋₄-alkyl or C₁₋₄-alkoxy or by a C₁₋₄-alkylene chain bonded to two adjacent carbon atoms; dioxolanylmethyl groups optionally substituted in the dioxolane ring by C₁₋₄-alkyl; C₂₋₆-alkanoyloxy-C₁₋₄-alkyl groups optionally substituted at the oxy-C₁₋₄-alkyl group by C₁₋₄-alkyl; double esters like 1-[[(C₁₋₄-alkyl)carbonyl]oxy]C₁₋₄-alkyl esters, e.g. (RS)-1-[[(isopropyl)-carbonyl]oxy]ethyl or (RS)-1-[[(ethyl)carbonyl]oxy]-2-methyl propyl (for preparation see e.g. F. W. Sum et al., Bioorg. Med. Chem. Lett. 9 (1999) 1921-1926 or Y. Yoshimura et al., The Journal of Antibiotics 39/9 (1986) 1329-1342 ); carbonate esters like 1-[[(C₄₋₇-cycloalkyloxy)carbonyl]oxy] C₁₋₄-alkyl esters, preferably (RS)-1-[[(cyclohexyloxy)carbonyl]oxy]ethyl (=cilexetil; for preparation see e.g. K. Kubo et al., J. Med. Chem. 36 (1993) 2343-2349, cited as “Kubo et al.” hereinafter)) or 2-oxo-1,3-dioxolan-4-yl-C₁₋₄-alkyl esters which optionally contain a double bond in the dioxolan ring, preferably 5-methyl-2-oxo-1,3-dioxolen-4-yl-methyl (=medoxomil, for preparation see e.g. Kubo et al.) or 2-oxo-1,3-dioxolan-4-yl-methyl (=(methyl)-ethylenecarbonate). Where the group forming a biolabile ester represents an optionally substituted phenyl-C₁₋₄-alkyl group, this may contain an alkylene chain with 1 to 3, preferably 1, carbon atoms and preferably stands for optionally substituted benzyl, in particular for 2-chlorobenzyl or 4-chlorobenzyl. Where the group forming a biolabile ester represents an optionally substituted phenyl group, the phenyl ring of which is substituted by a lower alkylene chain, this may contain 3 to 4, preferably 3, carbon atoms and in particular be indanyl. Where the group forming a biolabile ester represents an optionally substituted C₂₋₆-alkanoyloxy-C₁₋₄-alkyl group, the C₂₋₆-alkanoyl group may be straight-chain or branched.

R¹ preferably has the meanings hydrogen, C₁₋₄-alkyl, p-methoxybenzyl, N,N-di-(C₀₋₄-alkyl)amino-C₁₋₆-alkyl, (RS)-1-[[(isopropyl)carbonyl]oxy]ethyl, (RS)-1-[[(ethyl)carbonyl]oxy]-2-methylpropyl, (RS)-1-[[(cyclohexyloxy)carbonyl]oxy]ethyl, 5-methyl-2-oxo-1,3-dioxolen-4-yl-methyl, 2-oxo-1,3-dioxolan-4-yl-methyl or (RS)-1-[[(ethoxy)carbonyl]oxy]ethyl.

R² preferably has the meanings hydrogen, ethyl, methoxyethoxymethyl, (RS)-1-[[(isopropyl)carbonyl]oxy]ethyl, (RS)-1-[[(ethyl)carbonyl]oxy]-2-methylpropyl, (RS)-1-[[(cyclohexyloxy)carbonyl]oxy]ethyl, 5-methyl-2-oxo-1,3-dioxolen-4-yl-methyl, 2-oxo-1,3-dioxolan-4-yl-methyl or (RS)-1-[[(ethoxy)carbonyl]oxy]ethyl.

More preferred are the compounds which are selected from the group consisting of 2-[1-(1-carboxymethyl-2-oxo-2,3,4,5-tetrahydro-1H-benzo[b]azepin-3-ylcarbamoyl)-cyclopentylmethyl]-4-phenyl-butyric acid ethyl ester [alternative name: 3-[1-{2′-(ethoxycarbonyl)}-4′-phenylbutyl]-cyclopentan-1-carbonylamino]-2,3,4,5-tetrahydro-2-oxo-1H-1-benzazepin-1-acetic acid] of Formula II, 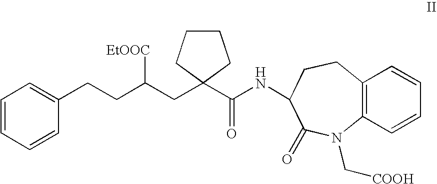
2-[1-(1-carboxymethyl-2-oxo-2,3,4,5-tetrahydro-1H-benzo[b]azepin-3-ylcarbamoyl)-cyclopentylmethyl]-4-naphthalen-1-yl-butyric acid ethyl ester [alternative name: 3-[1-{2-(ethoxycarbonyl)-4-(1-naphthyl)butyl]cyclopentyl}carbonyl)amino]-2-oxo-2,3,4,5-tetrahydro-1H-1-benzazepin-1-yl}acetic acid] of Formula III, 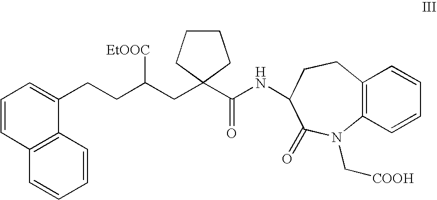
2-[1-(1-carboxymethyl-2-oxo-2,3,4,5-tetrahydro-1H-benzo[b]azepin-3-ylcarbamoyl)-cyclopentylmethyl]-4-phenyl-butyric acid of Formula IV, 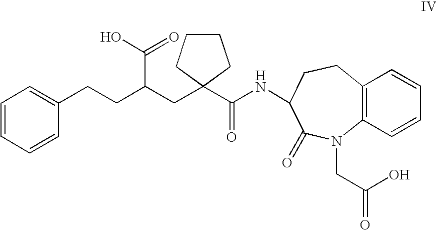
2-[1-(1-carboxymethyl-2-oxo-2,3,4,5-tetrahydro-1H-benzo[b]azepin-3-ylcarbamoyl)-cyclopentylmethyl]-4-naphthalen-1-yl-butyric acid of Formula V, 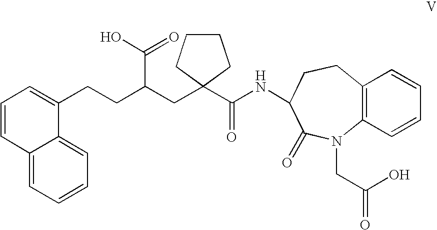
and physiologically compatible salts of the acids of Formulas II, III, IV and/or V. The compounds of Formulas II, III, IV and V are especially suited in their 3S,2′R forms. Most preferred is the compound of Formula II in its 3S,2′R form, also known as “daglutril” or “SLV306”. The compounds of Formula la are known, for example, from document EP 0 733 642 A1 which is incorporated herein by reference, and can be produced according to the production processes disclosed or referenced in this document or analogously to said production processes.

Further, compounds corresponding to Formula Ib, 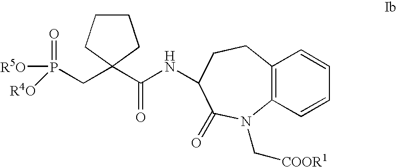
wherein R¹, R⁴ and R⁵ have the meanings given above, or physiologically compatible salts of acids of Formula Ib can be used as dually acting compounds capable of inhibiting NEP and the endogenous endothelin producing system. The compounds of Formula Ib are known, for example, from document EP 0 916 679 A1 which is incorporated herein by reference, and can be produced according to the production processes disclosed or referenced in this document or analogously to said production processes.

Suitable groups R¹ forming biolabile carboxylic acid esters in compounds of Formula Ib are those as specified for compounds of Formula Ia above.

Groups R⁴ and R⁵ suitable as groups forming biolabile phosphonic acid esters are those which can be removed under physiological conditions in vivo with release of the respective phosphonic acid function. For example, groups which are suitable for this purpose are lower alkyl groups, C₂-C₆-alkanoyloxymethyl groups optionally substituted on the oxymethyl group by lower alkyl, or phenyl or phenyl-lower alkyl groups whose phenyl ring is optionally mono- or polysubstituted by lower alkyl, lower alkoxy or by a lower alkylene chain bonded to two adjacent carbon atoms. If the group R⁴ and/or R⁵ forming a biolabile ester is or contains lower alkyl, this can be branched or unbranched and can contain 1 to 4 carbon atoms. If R⁴ and/or R⁵ are an optionally substituted alkanoyloxymethyl group, it can contain a preferably branched alkanoyloxy group having 2 to 6, preferably 3 to 5, carbon atoms and can, for example, be a pivaloyloxymethyl radical (=tert-butylcarbonyloxymethyl radical). If R⁴ and/or R⁵ are an optionally substituted phenyl-lower alkyl group, this can contain an alkylene chain having 1 to 3, preferably 1, carbon atoms. If the phenyl ring is substituted by a lower alkylene chain, this can contain 3 to 4, in particular 3, carbon atoms and the substituted phenyl ring is in particular indanyl.

The compounds of the formula Ib contain a chiral carbon atom, namely the carbon atom carrying the amide side chain in the 3-position of the benzazepine structure. The compounds can thus be present in two optically active stereoisomeric forms or as a racemate. The present invention includes both the racemic mixtures and the isomerically pure compounds of the formula I. If R⁴ and R⁵ in compounds of the formula Ib are not hydrogen and in each case have different meanings, the phosphorus atom of the phosphonic acid group can also be chiral. The invention also relates to the isomer mixtures and isomerically pure compounds of the formula Ib formed as a result of chiral phosphorus atoms.

When compounds of Formula Ib are used according to the invention, (3-{[1-(benzyloxy-ethoxy-phosphorylmethyl)-cyclopentanecarbonyl]-amino}-2-oxo-2,3,4,5-tetrahydro-benzo[b]azepin-1-yl)-acetic acid tert-butyl ester and isobutyric acid 1-[[1-(-1-carboxymethyl-2-oxo-2,3,4,5-tetrahydro-1H-benzo[b]azepin-3-ylcarbamoyl)-cyclopentylmethyl]-(1-isobutyryloxy-ethoxy)-phosphinoyloxy]-ethyl ester are preferred. Both of said compounds are particularly preferred when the stereochemistry at the chiral carbon atom (see above) is “S”, namely in their “(3S)” configuration. The compounds of Formula Ib are known, for example, from document EP 0 916 679 A1, and can be produced according to the production processes disclosed or referenced in this document or analogously to said production processes.

Also preferred are the compounds corresponding to Formula Ic, 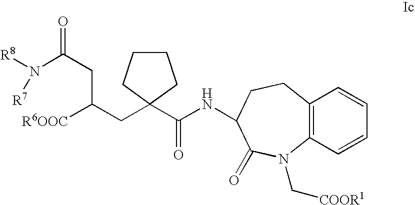
wherein R¹, R⁶, R⁷ and R⁸ have the above meanings, and physiologically compatible salts of acids of Formula Ic and/or physiologically compatible acid addition salts of compounds of Formula Ic, for the use as dually acting compounds capable of inhibiting NEP and the endogenous endothelin producing system in pharmacological compositions according to the invention. The compounds of Formula Ic are known, for example, from document WO 2005/030795 A1 which is incorporated herein by reference, and can be produced according to the production processes disclosed or referenced in this document or analogously to said production processes.

Where in compounds of Formula Ic the substituents R⁷ and/or R⁸ contain basic groups, in particular nitrogen, the compounds of Formula Ic may also occur in the form of acid addition salts. Physiologically compatible acid addition salts of compounds of Formula Ic are their conventional salts with inorganic acids, for example sulfuric acid, phosphoric acid or hydrohalic acids, preferably hydrochloric acid, or with organic acids, for example lower aliphatic monocarboxylic, dicarboxylic or tricarboxylic acids such as maleic acid, fumaric acid, tartaric acid, citric acid, or with sulfonic acids, for example lower alkanesulfonic acids such as methanesulfonic acid.

Suitable groups R¹ forming biolabile carboxylic acid esters in compounds of Formula Ic are those as specified for compounds of Formula Ia above. Suitable groups R⁶ forming biolabile carboxylic acid esters in compounds of Formula Ic are the same as specified for groups R² in compounds of Formula Ia above.

R⁷ preferably has the meanings hydrogen, methyl, ethyl, 2-hydroxyethyl or 3-hydroxypropyl, each hydroxyl group optionally being esterified with C₂₋₄-alkanoyl or an amino acid residue.

Where R⁸ has the meaning (C₀₋₄-alkyl)₂amino-C₁₋₆-alkyl, one or two C₀₋₄-alkyl groups can independently of each other be present. More specifically, “(C₀₋₄-alkyl)₂amino-C₁₋₆-alkyl” expressly comprises the meanings “(C₀)₂-alkylamino-C₁₋₆-alkyl”, “(C₀)(C₁₋₄)-alkylamino-C₁₋₆-alkyl” and “(C₁₋₄)₂-alkylamino-C₁₋₆-alkyl”. “(C₀)₂-alkylamino-C₁₋₆-alkyl” is meant to denominate an unsubstituted primary (═—NH₂) amino group bonded to C₁₋₆-alkyl(en); “(C₀)(C₁₋₄)-alkylamino-C₁₋₆-alkyl” is meant to denominate a secondary amino group monosubstituted by (C₁₋₄)-alkyl and bonded to C₁₋₆-alkyl(en); “(C₁₋₄)₂-alkylamino-C₁₋₆-alkyl” is meant to denominate a tertiary amino group disubstituted by (C₁₋₄)-alkyl and bonded to C₁₋₆-alkyl(en). R⁸ preferably has the meanings isopropyl; methoxyethyl; 2-hydroxyethyl or 3-hydroxypropyl, each hydroxyl group optionally being esterified with C₂₋₄-alkanoyl or an amino acid residue; 3-acetyloxy-n-propyl; cyclopropylmethyl; 2-methoxybenzyl, 4-methoxybenzyl; 4-methoxyphenylethyl; 2,4-dimethoxybenzyl; 1-naphthyl methyl; 3-oxo-1,1-dimethylbutyl; phenyl-2-oxoethyl; 2-(4-methoxyphenyl)-2-oxoethyl; 3-(2-oxoazepanyl); (C₀₋₄-alkyl)₂amino-C₁₋₆-alkyl, in particular dimethylamino-n-propyl, (methyl)aminoethyl, amino-n-propyl, amino-n-butyl or amino-n-pentyl.

Where R⁷ and R⁸ together are C₄₋₇-alkylene, the methylene groups of which are optionally replaced or optionally substituted, in each case morpholine; piperidine; 4-ketopiperidine; 4-hydroxypiperidine, optionally being esterified with C₂₋₄-alkanoyl or an amino acid residue at the hydroxyl group; piperazine or pyrrolidine is preferred.

Where in the compounds of Formula Ic hydroxyl groups are esterified with amino acid residues, these amino acid residues may be derived from natural or non-natural, □- or □-amino acids. Suitable amino acids which can be used are for example selected from the group cosisting of alanine, 2-aminohexanoic acid (=norleucine), 2-aminopentanoic acid (=norvaline), arginine, asparagine, aspartic acid, cysteine, 3,4-dihydroxyphenylalanine (=dopa), glutamine, glutamic acid, glycine, histidine, isoleucine, leucine, lysine, methionine, ornithine (=2,5-diaminovaleric acid), 5-oxo-2-pyrrolidinecarbonic acid (=pyroglutamic acid), phenylalanine, proline, serine, threonine, thyronine, tryptophan, tyrosine and valine. Preferred are amino acid residues which are derived from alanine, asparagine, glutamine, glycine, isoleucine, leucine, lysine, ornithine, phenylalanine, proline and valine.

The compounds of Formula Ic contain two chiral carbon atoms, namely the carbon atom bearing the amide side chain in position 3 of the benzazepine skeleton (=C_{b}*) and the carbon atom bearing the radical “—COOR⁶” (=Cₐ*). The compounds can thus be present in a total of four stereoisomeric forms. The present invention comprises both the mixtures of stereoisomers and enantiomers, and also the isomerically pure compounds of Formula Ic. Isomerically pure compounds of Formula Ic are preferred. Particularly preferred are compounds of Formula Ic wherein the carbon atom bearing the amide side chain in position 3 of the benzazepine skeleton is in the “S” configuration. With respect to the chiral carbon atom “Cₐ” bearing the radical “—COOR⁶”, the configuration of the compounds of Formula I which is preferred according to the invention in the context of this invention is provisionally assigned the configuration designation “rel1”. It can be derived by analogous observations of suitable compounds of known configuration that the preferred configuration “rel1” at the chiral centre “Cₐ” is probably likewise the “S” configuration.

Particularly preferred compounds of Formula Ic are selected from-the group consisting of:
2-{[1-({[1-(carboxymethyl)-2-oxo-2,3,4,5-tetrahydro-1H-1-benzazepin-3-yl]amino}-carbonyl)cyclopentyl]methyl}-4-[isopropyl(methyl)amino]-4-oxobutanoic acid;
2-{[1-({[1-(carboxymethyl )-2-oxo-2,3,4,5-tetrahydro-1H-1-benzazepin-3-yl]amino}-carbonyl)cyclopentyl]methyl}-4-(dimethylamino)-4-oxobutanoic acid;
2-{[1-({[1-(carboxymethyl)-2-oxo-2,3,4,5-tetrahydro-1H-1-benzazepin-3-yl]amino}-carbonyl)cyclopentyl]methyl}-4-(diethylamino)-4-oxobutanoic acid;
2-{[1-({[1-(carboxymethyl )-2-oxo-2,3,4,5-tetrahydro-1H-1-benzazepin-3-yl]amino}-carbonyl)cyclopentyl]methyl}-4-[(2-hydroxyethyl)(methyl)amino]-4-oxobutanoic acid;
2-{[1-({[1-(carboxymethyl)-2-oxo-2,3,4,5-tetrahydro-1H-1-benzazepin-3-yl]amino}-carbonyl)cyclopentyl]methyl}-4-[(3-hydroxypropyl)(methyl)amino]-4-oxobutanoic acid;
2-{[1-({[1-(carboxymethyl)-2-oxo-2,3,4,5-tetrahydro-1H-1-benzazepin-3-yl]amino}-carbonyl)cyclopentyl]methyl}-4-(4-hydroxypiperidin-1-yl )-4-oxobutanoic acid;
2-{[1-({[1-(carboxymethyl)-2-oxo-2,3,4,5-tetrahydro-1H-1-benzazepin-3-yl]amino}-carbonyl)cyclopentyl]methyl}-4-oxo-4-[4-(L-valyloxy)piperidin-1-yl]butanoic acid;
2-{[1-({[1-(carboxymethyl )-2-oxo-2,3,4,5-tetrahydro-1H-1-benzazepin-3-yl]amino}-carbonyl)cyclopentyl]methyl}-4-morpholin-4-yl-4-oxobutanoic acid;
2-{[1-({[1-(carboxymethyl)-2-oxo-2,3,4,5-tetrahydro-1H-1-benzazepin-3-yl]amino}-carbonyl)cyclopentyl]methyl}-4-oxo-4-(4-oxopiperidin-1-yl)butanoic acid;
4-[bis(2-hydroxyethyl)amino]-2-{[1-({[1-(carboxymethyl)-2-oxo-2,3,4,5-tetrahydro-1H-1-benzazepin-3-yl]amino}carbonyl)cyclopentyl]methyl}-4-oxobutanoic acid;
2-{[1-({[1-(carboxymethyl)-2-oxo-2,3,4,5-tetrahydro-1H-1-benzazepin-3-yl]amino}-carbonyl)cyclopentyl]methyl}-4-{ethyl[3-(ethylamino)propyl]amino}-4-oxobutanoic acid;
2-{[1-({[1-(carboxymethyl)-2-oxo-2,3,4,5-tetrahydro-1H-1-benzazepin-3-yl]amino}-carbonyl)cyclopentyl]methyl}-4-[[2-(dimethylamino)ethyl](methyl)amino]-4-oxobutanoic acid;
4-[(3-aminopropyl)(ethyl)amino]-2-{[1-({[1-(carboxymethyl)-2-oxo-2,3,4,5-tetrahydro-1H-1-benzazepin-3-yl]amino}carbonyl)cyclopentyl]methyl}-4-oxobutanoic acid,
2-{[1-({[1-(carboxymethyl)-2-oxo-2,3,4,5-tetrahydro-1 H-1-benzazepin-3-yl]amino}-carbonyl)cyclopentyl]methyl}-4-{methyl [2-(methylamino)ethyl]amino}-4-oxobutanoic acid;
4-[(4-aminobutyl)(methyl)amino]-2-{[1-({[1-(carboxymethyl)-2-oxo-2,3,4,5-tetrahydro-1H-1-benzazepin-3-yl]amino}carbonyl)cyclopentyl]methyl}-4-oxobutanoic acid;
4-[(4-aminobutyl)(ethyl)amino]-2-{[1-({[1-(carboxymethyl)-2-oxo-2,3,4,5-tetrahydro-1H-1-benzazepin-3-yl]amino}carbonyl)cyclopentyl]methyl}-4-oxobutanoic acid;
2-{[1-({[1-(carboxymethyl)-2-oxo-2,3,4,5-tetrahydro-1H-1-benzazepin-3-yl]amino}-carbonyl)cyclopentyl]methyl}-4-{methyl[3-(methylamino)propyl]amino}-4-oxobutanoic acid, and
4-[(5-aminopentyl)(methyl)amino]-2-{[1-({[1-(carboxymethyl)-2-oxo-2,3,4,5-tetrahydro-1H-1-benzazepin-3-yl]amino}carbonyl)cyclopentyl]methyl}-4-oxobutanoic acid,
   together with their biolabile esters and physiologically compatible salts of acids of these compounds of Formula Ic and/or physiologically compatible acid addition salts of these compounds of Formula Ic.

Phosphodiesterase 5 terminates the cellular actions of the second messenger molecule cyclic GMP. PDE V inhibitors will therefore increase and prolong the actions of endogenous substances that signal via the cyclic GMP pathway, including nitric oxide released as a neurotransmifter from nitrergic nerves. To date, the most widely used PDE V inhibitors have proved vital in the elucidation of the widespread role of cyclic GMP in nitrergic transmission and have provided a major breakthrough in the treatment of erectile dysfunction in men. PDE V inhibitors which can be used according to the present invention may be selected from the group consisting of avanafil, AWD-12-250, BF/GP-385, BMS-22313, BMS-341400, CP-248, CP-461, DA-8159, dasantafil, DMPPO, E-4021, E-8010, EMD-82639, EMR-62203, exisulind, FR-181074, FR-226807, FR-229934, GF-248, KF-31327, KT-734, LAS-34179, lusupultide, MJ-12504, NCX-911, NM-702, OPC-35564, OSI-461, QAD-171A, roflumilast, SB-96231, Sch-46642, Sch-51866, Sch-59498, sildenafil, sildenafil citrate, SK-350, SK-3530, SKF-96231, sophoflavescenol, SR-265579, T-0156, T-1032, tadalafil, UK-114502, UK-114542, UK-357903, UK-369003, UK-371800, UK-83405, vardenafil, WIN-65579, WS-63967, YC-1 and zaprinast which are all known per se, or any physiologically compatible salts, solvates, prodrugs or esters thereof.

Preferred PDE V inhibitors are selected from the group consisting of avanafil, dasantafil, sildenafil, sildenafil citrate, tadalafil, vardenafil and zaprinast, or any physiologically compatible salts, solvates, prodrugs or esters thereof. Sildenafil, sildenafil citrate, tadalafil and vardenafil are more preferred PDE V inhibitors.

The pharmaceutical compositions according to the invention can be prepared in a manner known per se and thus can be obtained as formulations suitable for enteral, such as oral or rectal, or parenteral administration to mammals or humans, comprising a therapeutical effective amount of the pharmacologically active agents, alone or in combination with one or more pharmaceutically acceptable auxiliaries and/or carriers, especially suitable for enteral or parenteral application. Pharmaceutical compositions for enteral or parenteral administration are, for example, in unit dosage forms, such as coated tablets, tablets, capsules or suppositories and also ampoules. These are prepared in a manner which is known per se, for example using conventional mixing, granulation, coating, solubulizing or lyophilizing processes. Typical oral formulations include coated tablets, tablets, capsules, syrups, elixirs and suspensions. Capsules may contain the active agents e.g. in form of powders, granules, pellets, beadlets or microtablets. For example, a pharmaceutical composition according to the invention may consist of from about 0.1% to 90%, preferably of from about 1% to about 80%, of the active agents, the rest being made up by pharmaceutically acceptable auxiliaries and/or carriers. Thus, pharmaceutical compositions for oral use can be obtained by combining the active compounds with solid excipients, if desired granulating a mixture which has been obtained, and, if required or necessary, processing the mixture or granulate into tablets or coated tablet cores after having added suitable auxiliary substances. Typical injectable formulations include solutions and suspensions.

In one embodiment of the pharmaceutical compositions according to the invention, the active agents (a), (b) and (c) can be obtained and administered together, e.g. in one combined unit dosage form like in one tablet or capsule, i.e. in a physical combination. In such a combined unit dosage form, the different active agents (a), (b) and (c) can be segregated from each other, e.g. by means of different layers in said tablet, e.g. by the use of inert intermediate layers known in the art; or by means of different compartments in said capsule. The corresponding active agents or their pharmaceutically acceptable salts may also be used in form of their hydrates or include other solvents used for crystallization. A unit dosage form may be a fixed combination. A unit dosage form, in particular a fixed combination of the active agents (a), (b) and (c) is a preferred alternative of this embodiment. Fixed combinations comprising daglutril and sildenafil or sildenafil citrate, daglutril and tadalafil, daglutril and vardenafil or daglutril and zaprinast are preferred embodiments of the invention.

In another embodiment the active agents (a), (b) and (c) can be obtained and administered in two or more separate unit dosage forms, e.g. in two or more tablets or capsules, the tablets or capsules being physically segregated from each other. The two or more separate unit dosage forms can be administered simultaneously or stepwise (separately), e.g. sequentially one after the other in either order. Thus, the active agents can be administered in either order at the same time or at different times spread over the day, the optimal dosage regimen usually being determined by prescription of a physician. When a dually acting compound capable of inhibiting NEP and the endogenous endothelin producing system is used to embody the combination of active agents (a) and (b), the active agents [(a)+(b)] and (c) in the pharmaceutical composition can favourably be present in two separate dosage forms, usually complementary or balanced for combined use, e.g. as two different tablets or capsules, usually further comprising pharmaceutically acceptable auxiliaries and/or carriers, or in different compartments of one single capsule. Thus, in this embodiment at least the PDE V inhibitor is present in a unit single dosage form physically segregated from the other active agent(s).

The typical pharmaceutically acceptable auxiliaries and/or carriers for use in the formulations described above are exemplified by: sugars such as lactose, sucrose, mannitol and sorbitol; starches such as cornstarch, tapioca starch and potato starch; cellulose and derivatives such as sodium carboxymethyl cellulose, ethyl cellulose and methyl cellulose; calcium phosphates such as dicalcium phosphate and tricalcium phosphate; sodium sulfate; calcium sulfate; polyvinylpyrrolidone; polyvinyl alcohol; stearic acid; alkaline earth metal stearates such as magnesium stearate and calcium stearate; stearic acid; vegetable oils such as peanut oil, cottonseed oil, sesame oil, olive oil and corn oil; non-ionic, cationic and anionic surfactants; ethylene glycol polymers; betacyclodextrin; fatty alcohols; and hydrolyzed cereal solids, as well as other non-toxic compatible fillers, binders, disintegrants, agents, e.g. talcum; buffers, preservatives, antioxidants, lubricants, flavoring and the like commonly used in pharmaceutical formulations.

In a specific embodiment of said first aspect, the invention also relates to a kit comprising in separate containers in a single package pharmaceutical dosage forms for use in combination, comprising,
i1) in one separate container a pharmaceutical dosage form comprising at least one neutral endopeptidase inhibitor and in a second separate container a pharmaceutical dosage form comprising at least one inhibitor of the endogenous endothelin producing system, or
i2) in one separate container a pharmaceutical dosage form comprising a dually acting compound capable of inhibiting neutral endopeptidase and the endogenous endothelin producing system, and
ii) in another separate container a pharmaceutical dosage form comprising at least one PDE V inhibitor.

The kit form is particularly advantageous but not limited to the case when the separate components must be administered in different dosage forms or are administered at different dosage intervals. The dosage forms may favourably be oral formulations like tablets or capsules. The separate containers may e.g. be blister packs—in particular where the oral formulations are tablets or coated tablets, boxes or other containers commonly used to package pharmaceutical dosage forms. Preferred are alternatives of the kit which comprise in one separate container a pharmaceutical dosage form comprising a dually acting compound capable of inhibiting neutral endopeptidase and the endogenous endothelin producing system, and in another separate container a pharmaceutical dosage form comprising at least one PDE V inhibitor. The kit may further comprise leaflets or other written instructions as to how the different kit constituents may best be used in order to achieve best therapeutic results with the provided combination of active ingredients.

In a second aspect, the invention also relates to a use of at least one NEP-inhibitor in combination with at least one inhibitor of the endogenous endothelin producing system and at least one PDE V inhibitor, for the preparation of a pharmaceutical composition or medicament for the inhibition or treatment of sexual dysfunction and/or endothelial dysfunction and/or a cardiovascular disease in mammals and humans.

The sexual dysfunction may be a female sexual dysfunction or a male sexual dysfunction. Treatment or inhibition of male sexual dysfunction is preferred. Where the sexual dysfunction is male sexual dysfunction, the combination therapy of the present invention is particularly suited to treat or prevent erectile dysfunction, ejaculatory disorders and/or desire disorders. Most preferred is the treatment or prevention of erectile dysfunction.

The cardiovascular disease may be selected from angina pectoris; angina abdominalis; arrhythmias; cardiac hypertrophy; congestive heart failure; coronary heart disease; hypertension, in particular essential hypertension, pulmonary hypertension, renal hypertension and/or hypertension associated with obesity, insulin resistance and/or diabetes; myocardial infarction; restenosis and/or stroke. In particular, the cardiovascular disease may beselected from essential hypertension, pulmonary hypertension, renal hypertension, hypertension associated with obesity and/or congestive heart failure.

In a third aspect, the invention relates to a method of treating or inhibiting sexual dysfunction and/or endothelial dysfunction and/or a cardiovascular disease in mammals and humans, comprising administering to a subject in need thereof an effective amount of a combination of at least one NEP-inhibitor, at least one inhibitor of the endogenous endothelin producing system and at least one PDE V inhibitor. Subjects in need of such treatments are in particular those humans or mammals who are suffering from or being susceptible to sexual dysfunction and/or endothelial dysfunction and/or a cardiovascular disease. The active agents (a), (b) and (c) can be obtained and administered together, sequentially one after the other or separately in one combined unit dosage form, e.g. in one tablet or capsule. Thus, the active agents can be administered in either order at the same time or at different times spread over the day, the optimal dosage regimen usually being determined by prescription of a physician.

In one specific embodiment of said third aspect, a fixed combination of a dually acting compound capable of inhibiting neutral endopeptidase and the endogenous endothelin producing system, and an PDE V inhibitor can be used. Fixed combinations comprising daglutril and sildenafil or sildenafil citrate, daglutril and tadalafil, daglutril and vardenafil or daglutril and zaprinast are preferred alternatives of this specific embodiment.

Description of the Pharmacological Test Methods

The beneficial effects of the combination therapy according to the invention can e.g. be shown in the following test models:

1. Big-ET-Induced Hypertension Following Intravenous Bolus Administration in Rats

Male Sprague-Dawley rats (˜250 body weight, fed a standard chow diet, fasted for 18 h prior to the experiments, with free access to drinking water) were anaesthetised with 1 ml/kg Rompun/Ketavet 1:1, and then transferred to servo-controlled, heated tables to maintain body temperature at 37° C. A pressure transducer (Statham) was connected to one carotid artery for measurement of arterial blood pressure. Both jugular veins were cannulated for compound and Big-ET-infusion (Big-ET=endogenous precursor of endothelin), respectively. After an equilibration period of 20 minutes (=min), the animals received either vehicle (10% arlatone G, 10% ethanol, 80% H₂O), sildenafil (1 mg/kg), compound of Formula IV (10 μmol/kg), or a combination of sildenafil+compound of Formula IV intravenously (over 1 min in a total volume of 2 ml/kg body weight). 8 min later, 0.5 nmol/kg Big-ET was infused (over 1 min; Big-ET was from Bachem, Heidelberg). Systolic (SAP) and diastolic arterial blood pressures and heart rate were recorded every 5 min for the next 15 min.

The maximal pressor and bradycardic effects were typically reached within 5 min following Big-ET administration.

Immediately after administration (and before the Big-ET challenge), sildenafil produced a drop in blood pressure (by 15±3 mm Hg systolic blood pressure as compared to baseline) which was stable for at least 8 min (i.e. until the timepoint of Big-ET application). This hypotension was not changed when sildenafil was administered in combination with the compound of Formula IV (lowering of blood pressure by 14±4 mm Hg vs. baseline). The compound of Formula IV did thus not reinforce the hypotensive action of sildenafil.

TABLE 1
Effects of intravenous bolus administration of sildenafil,
compound of Formula IV, and combination on maximal
Big-ET-induced pressor response

| | Maximal Big-ET-induced | | |
|---|---|---|---|
| Compound | increase in SAP [mm Hg] | SEM | n |
| Vehicle | 66.0 | 5.5 | 6 |
| Sildenafil (1 mg/kg) | 42.6 | 5.1 | 5 |
| compound of Formula IV | 43.0 | 8.0 | 6 |
| (10 μmol/kg) | | | |
| Sildenafil + compound of | 21.3 | 4.1 | 8 |
| Formula IV | | | |

The results are expressed as mean±SEM of the maximal systolic blood pressure increase (from baseline) observed after the Big-ET challenge (n: number of animals).
2. Effect on Blood Pressure in Spontaneously Hypertensive Rats

Male spontaneously hypertensive rats (=SHR, insulin resistant strain from Charles River; aged 6 months) were equipped with telemetry transmitters for continuous monitoring of blood pressure and heart rate. Telemetry transmitters for continuous monitoring of blood pressure, heart rate and locomotor activity (TA11PA-C40, Data Sciences, USA) were implanted intraabdominally under inhalative halothane anesthesia. A midline abdominal incision was made, and the abdominal aorta was visualized by removal of retroperitoneal fat and connective tissue. A ligature was placed caudal of the renal arteries, the aorta was punctured with a 22G needle, and the catheter was advanced into the aorta. The entry point was sealed with tissue adhesive (Vetbond®, 3M, USA), the ligature was removed, and the abdominal incision was closed. Measurements of aortic pressure were taken every 5 min for 4 seconds (=s) each at a sampling rate of 500 Hz, and were corrected for the corresponding ambient pressure (ambient pressure monitor, C11PR, Data Sciences, USA).

On the day of the experiments, blood pressure was first monitored for 30 min under baseline (untreated) conditions before a group of animals received sildenafil (3 mg/kg), and another one sildenafil (3 mg/kg) combined with compound of Formula IV (10 μmol/kg) via injection into the caudal vein. Blood pressure was then measured for another 30 min. On the next day, the groups were treated in a cross-over mode, i.e. that—after the 30 min baseline measurement period—the ones that had been treated with sildenafil alone on the previous day were given the combination, and the ones treated with the combination on the day before now received sildenafil alone.

The blood pressure (as well as heart rate and activity values), sampled in 5 min intervals by the Dataquest system, were used for calculation of individual means, i.e. the average blood pressure over the 30 min baseline period, and the average value over the last 15 min of the 30 min interval following the intravenous treatment with compounds.

Pretreatment with compound of Formula IV (intravenous bolus, 30 min before sildenafil) caused an even stronger potentiation of the antihypertensive effect of sildenafil (decrease in systolic blood pressure by 33±14 mm Hg).

TABLE 2
Effects of intravenous bolus administration of sildenafil, compound of
Formula IV, and combination on systolic blood pressure in SHR

| | Average change in | | |
|---|---|---|---|
| | systolic blood | | |
| Compound | pressure [mm Hg] | SEM | n |
| Sildenafil (3 mg/kg) | −10.5 | 2.9 | 6 |
| Sildenafil + compound | −19.3 | 4.9 | 5 |
| of Formula IV | | | |

Shown are means values±SEM of the change vs. baseline in systolic blood pressure after compound administration (as averaged over the last 15 min of the 30 min observation period following treatment); n: number of animals.

The dosage of the active agents can depend on a variety of factors, such as mode of administration, species, age and/or individual condition. Suitable dosages for the active agents of the pharmaceutical combination according to the present invention are therapeutically effective dosages, for example those which are commercially available. Normally, in the case of oral administration, an approximate daily dose of from about 5 mg to about 600 mg is to be estimated for each of the active agents e.g. for a patient of approximately 75 kg in weight. For example, a pharmaceutical composition according to the invention may preferably comprise daglutril as dually acting compound capable of inhibiting ECE and hSEP in the range of 5-600 mg. The dose range of PDE V inhibitors which are present in the pharmaceutical compositions according to the invention may vary depending on i.a. the substance used and may be. The administration of the pharmaceutical composition may occur up to three times a day. Once daily administration forms are preferred.

### EXAMPLE I

### Capsules Containing Daglutril and Sildenafil Citrate

Capsules with the following composition per capsule are produced:

| | |
|---|---|
| Daglutril tricalcium phosphate salt | 200 mg |
| Sildenafil citrate | 50 mg |
| Corn starch | 50 mg |
| Lactose | 80 mg |
| Ethyl acetate | q.s. |

The active agents, the corn starch and the lactose are processed into a homogeneous pasty mixture using ethyl acetate. The paste is ground and the resulting granules are placed on a suitable tray and dried at 45° C. in order to remove the solvent. The dried granules are passed through a crusher and mixed in a mixer with the further following auxiliaries:

| | |
|---|---|
| Talcum | 5 mg |
| Magnesium stearate | 5 mg |
| Corn starch | 9 mg |

and are then poured into 400 mg capsules (=capsule size 0).

The foregoing description and examples have been set forth merely to illustrate the invention and are not intended to be limiting. Since modifications of the described embodiments incorporating the spirit and substance of the invention may occur to persons skilled in the art, the invention should be construed broadly to include all variations within the scope of the appended claims and equivalents thereof.

1. A pharmaceutical composition comprising in combination a pharmacologically effective quantity of each of
a) at least one NEP-inhibitor,
b) at least one inhibitor of the endogenous endothelin producing system, and
c) at least one PDE V inhibitor.

2. A pharmaceutical composition according to claim 1, further comprising at least one pharmaceutically acceptable auxiliary or carrier.

3. A pharmaceutical composition according to claim 1, wherein said composition is in orally administrable form.

4. A pharmaceutical composition according to claim 3, wherein said orally administrable form is selected from the group consisting of tablets, coated tablets, capsules, syrups, elixirs, suspensions, and combinations thereof.

5. A pharmaceutical composition according to claim 1, wherein the PDE V inhibitor is present in a unit single dosage form physically segregated from other active agents.

6. A pharmaceutical composition according to claim 1, wherein the inhibitor of the endogenous endothelin producing system is selected from the group consisting of inhibitors of endothelin converting enzyme, inhibitors of human soluble endopeptidase, and dually acting compounds capable of inhibiting endothelin converting enzyme and human soluble endopeptidase.

7. A pharmaceutical composition according to claim 1, wherein the neutral endopeptidase inhibitor (a) and the inhibitor of the endogenous endothelin producing system (b) are present together in the form of a single, dually acting compound which inhibits both neutral endopeptidase and the endogenous endothelin producing system.

8. A pharmaceutical composition according to claim 7, wherein said dually acting compound inhibits both neutral endopeptidase and human soluble endopeptidase.

9. A pharmaceutical composition according to claim 7, wherein said dually acting compound which inhibits both neutral endopeptidase and the endogenous endothelin producing system is a compound corresponding to Formula I, 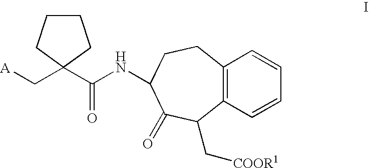
wherein
R¹ is hydrogen or a group forming a biolabile carboxylic acid ester,
A represents a group selected from the subgroups:
(a), 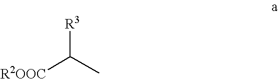
wherein
R² is hydrogen or a a group forming a biolabile carboxylic acid ester, and ^{R}³ is a phenyl-C₁₋₄-alkyl group which can optionally be substituted in the phenyl ring by C₁₋₄-alkyl, C₁₋₄-alkoxy or halogen; or a naphthyl-C₁₋₄-alkyl group;
(b), 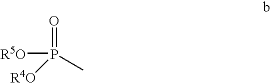
wherein
R⁴ is hydrogen or a group forming a biolabile phosphonic acid ester, and
R⁵ is hydrogen or a group forming a biolabile phosphonic acid ester; and (c), 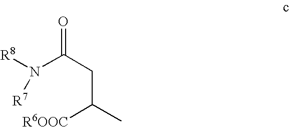
wherein
R⁶ is hydrogen or a group forming a biolabile carboxylic acid ester,
R⁷ is hydrogen, C₁₋₄-alkyl or C₁₋₄-hydroxyalkyl, the hydroxyl group of which is optionally esterified with C₂₋₄-alkanoyl or an amino acid residue, and
R⁸ is C₁₋₄-alkyl; C₁₋₄-alkoxy-C₁₋₄-alkyl; C₁₋₄-hydroxyalkyl, which is optionally substituted by a second hydroxyl group, and the hydroxyl groups of which are each optionally esterified with C₂₋₄-alkanoyl or an amino acid residue; (C₀₋₄-alkyl)₂amino-C₁₋₆-alkyl; C₃₋₇-cycloalkyl; C₃₋₇-cycloalkyl-C₁₋₄-alkyl; phenyl-C₁₋₄-alkyl, the phenyl group of which is optionally substituted 1-2 times by C₁₋₄-alkyl, C₁₋₄-alkoxy and/or halogen; naphthyl-C₁₋₄-alkyl; C₃₋₆-oxoalkyl; phenylcarbonylmethyl, the phenyl group of which is optionally substituted one or two times by C₁₋₄-alkyl, C₁₋₄-alkoxy and/or halogen, or 2-oxoazepanyl, or
R⁷ and R⁸ together are C₄₋₇-alkylene, the methylene groups of which are optionally replaced one or two times by carbonyl, nitrogen, oxygen and/or sulfur and which are optionally substituted once by hydroxy, which is optionally esterified with C₂₋₄-alkanoyl or an amino acid residue; C₁₋₄-alkyl; C₁₋₄-hydroxyalkyl, the hydroxyl group of which is optionally esterified with C₂₋₄-alkanoyl or an amino acid residue; phenyl or benzyl;
or a physiologically compatible salt thereof.

10. A pharmaceutical composition according to claim 9, wherein said dually acting compound which inhibits both neutral endopeptidase and the endogenous endothelin producing system is a compound corresponding to Formula Ia, 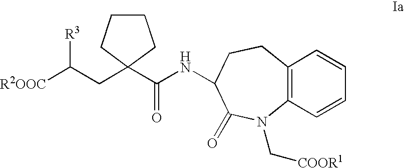
wherein
R¹ is hydrogen or a group forming a biolabile carboxylic acid ester,
R² is hydrogen or a a group forming a biolabile carboxylic acid ester, and
R³ is a phenyl-C₁₋₄-alkyl group which can optionally be substituted in the phenyl ring by C₁₋₄-alkyl, C₁₋₄-alkoxy or halogen; or a naphthyl-C₁₋₄-alkyl group;
or a physiologically compatible salt thereof.

11. A pharmaceutical composition according to claim 10, wherein said dually acting compound which inhibits both neutral endopeptidase and the endogenous endothelin producing system is selected from the group consisting of:
2-[1-(1-Carboxymethyl-2-oxo-2,3,4,5-tetrahydro-1H-benzo[b]azepin-3-ylcarbamoyl)-cyclopentylmethyl]-4-phenyl-butyric acid ethyl ester;
2-[1-(1-Carboxymethyl-2-oxo-2,3,4,5-tetrahydro-1H-benzo[b]azepin-3-ylcarbamoyl)-cyclopentylmethyl]-4-naphthalen-1-yl-butyric acid ethyl ester;
2-[1-(1-Carboxymethyl-2-oxo-2,3,4,5-tetrahydro-1H-benzo[b]azepin-3-ylcarbamoyl)-cyclopentylmethyl]-4-phenyl-butyric acid;
2-[1-(1-carboxymethyl-2-oxo-2,3,4,5-tetrahydro-1H-benzo[b]azepin-3-ylcarbamoyl)-cyclopentylmethyl]-4-naphthalen-1-yl-butyric acid;
and physiologically compatible salts thereof.

12. A pharmaceutical composition according to claim 9, wherein said dually acting compound which inhibits both neutral endopeptidase and the endogenous endothelin producing system is a compound corresponding to Formula Ic, 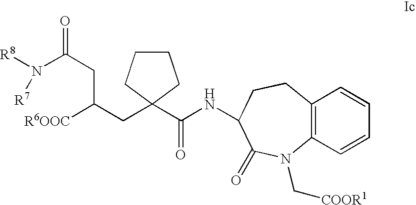
wherein
R⁶ is is hydrogen or a group forming a biolabile ester,
R⁷ is hydrogen, C₁₋₄-alkyl or C₁₋₄-hydroxyalkyl, the hydroxyl group of which is optionally esterified with C2-4-alkanoyl or an amino acid residue, and
R⁸ is C₁₋₄-alkyl; C₁₋₄-alkoxy-C₁₋₄-alkyl; C₁₋₄-hydroxyalkyl, which is optionally substituted by a second hydroxyl group and the hydroxyl groups of which are each optionally esterified with C₂₄-alkanoyl or an amino acid residue; (C₀₋₄-alkyl)₂amino-C₁₋₆-alkyl; C₃₋₇-cycloalkyl; C₃₋₇-cycloalkyl-C₁₋₄-alkyl; phenyl-C₁₋₄-alkyl, the phenyl group of which is optionally substituted 1-2 times by C₁₋₄-alkyl, C₁₋₄-alkoxy and/or halogen; naphthyl-C₁₋₄-alkyl; C₃₋₆-oxoalkyl; phenylcarbonylmethyl, the phenyl group of which is optionally substituted 1-2 times by C₁₋₄-alkyl, C₁₋₄-alkoxy and/or halogen, or 2-oxoazepanyl, or
R⁷ and R⁸ together are C₄₋₇-alkylene, the methylene groups of which are optionally replaced 1-2 times by carbonyl, nitrogen, oxygen and/or sulfur and which are optionally substituted once by hydroxy, which is optionally esterified with C₂₋₄-alkanoyl or an amino acid residue; C₁₋₄-alkyl; C₁₋₄-hydroxyalkyl, the hydroxyl group of which is optionally esterified with C₂₋₄-alkanoyl or an amino acid residue; phenyl or benzyl;
or a physiologically compatible salt thereof.

13. A pharmaceutical composition according to claim 12, wherein R⁷ denotes hydrogen, methyl, ethyl, 2-hydroxyethyl or 3-hydroxypropyl, each hydroxyl group optionally being esterified with C₂₋₄-alkanoyl or an amino acid residue.

14. A pharmaceutical composition according to claim 12, wherein R⁸ denotes isopropyl; methoxyethyl; 2-hydroxyethyl or 3-hydroxypropyl, each hydroxyl group optionally being esterified with C₂₋₄-alkanoyl or an amino acid residue; 3-acetyloxy-n-propyl; cyclopropylmethyl; 2-methoxybenzyl, 4-methoxybenzyl; 4-methoxyphenylethyl; 2,4-dimethoxybenzyl; 1-naphthylmethyl; 3-oxo-1,1-dimethylbutyl; phenyl-2-oxoethyl; 2-(4-methoxyphenyl)-2-oxoethyl; 3-(2-oxoaze-panyl); (C₀₋₄-alkyl)₂amino-C₁₋₆-alkyl.

15. A pharmaceutical composition according to claim 12, wherein R⁸ denotes dimethylamino-n-propyl, (methyl)aminoethyl, amino-n-propyl, amino-n-butyl, or amino-n-pentyl.

16. A pharmaceutical composition according to claim 1, wherein said at least one PDE V inhibitor is selected from the group consisting of avanafil, AWD-12-250, BF/GP-385, BMS-22313, BMS-341400 , CP-248, CP-461, DA-8159, dasantafil, DMPPO, E-4021, E-8010, EMD-82639, EMR-62203, exisulind, FR-181074, FR-226807, FR-229934, GF-248, KF-31327, KT-734, LAS-34179, lusupultide, MJ-12504, NCX-911, NM-702, OPC-35564, OSI-461, QAD-171A, roflumilast, SB-96231, Sch-46642, Sch-51866, Sch-59498, sildenafil, sildenafil citrate, SK-350, SK-3530, SKF-96231, sophoflavescenol, SR-265579, T-0156, T-1032, tadalafil, UK-1 14502, UK-1 14542, UK-357903, UK-369003, UK-371800, UK-83405, vardenafil, WIN-65579, WS-63967, YC-1, zaprinast, and physiologically compatible salts, solvates, prodrugs and esters thereof.

17. A pharmaceutical composition according to claim 1, wherein said at least one PDE V inhibitor is selected from the group consisting of avanafil, dasantafil, sildenafil, sildenafil citrate, tadalafil, vardenafil, zaprinast, and physiologically compatible salts, solvates, prodrugs and esters thereof.

18. A pharmaceutical composition according to claim 1, wherein said at least one PDE V inhibitor is selected from the group consisting of sildenafil, sildenafil citrate, tadalafil, vardenafil, and physiologically compatible salts, solvates, prodrugs and esters thereof.

19. A method of treating or inhibiting an endothelial dysfunction in a human or other mammal subject in need thereof, said method comprising administering to said subject an effective amount of a combination of (a) at least one neutral endopeptidase inhibitor, (b) at least one inhibitor of the endogenous endothelin producing system, and (c) at least one PDE V inhibitor.

20. A method of treating or inhibiting a sexual dysfunction in a human or other mammal subject in need thereof, said method comprising administering to said subject an effective amount of a combination of at least one neutral endopeptidase inhibitor, at least one inhibitor of the endogenous endothelin producing system, and at least one PDE V inhibitor.

21. A method according to claim 20, wherein said sexual dysfunction is a female sexual dysfunction.

22. A method according to claim 20, wherein said sexual dysfunction is a male sexual dysfunction.

23. A method according to claim 22, wherein said male sexual dysfunction is selected from the group consisting of erectile dysfunction, ejaculatory disorders and desire disorders.

24. A method according to claim 23, wherein the sexual dysfunction is erectile dysfunction.

25. A method of treating or inhibiting a cardiovascular disease in a human or other mammal subject in need thereof, said method comprising administering to said subject an effective amount of a combination of at least one neutral endopeptidase inhibitor, at least one inhibitor of the endogenous endothelin producing system, and at least one PDE V inhibitor.

26. A method according to claim 25, wherein said cardiovascular disease is selected from the group consisting of angina pectoris, angina abdominalis, arrhythmias, cardiac hypertrophy, congestive heart failure, coronary heart disease, hypertension, myocardial infarction, restenosis, and stroke.

27. A method according to claim 25, wherein said cardiovascular disease is selected from the group consisting of essential hypertension, pulmonary hypertension, renal hypertension, and hypertension associated with obesity, insulin resistance or diabetes.

28. Method according to claim 25, wherein said cardiovascular disease is selected from the group consisting of essential hypertension, pulmonary hypertension and congestive heart failure.

29. A kit comprising in separate containers in a single package pharmaceutical dosage forms for use in combination, comprising:
i1) in one separate container a pharmaceutical dosage form comprising at least one neutral endopeptidase inhibitor and in a second separate container a pharmaceutical dosage form comprising at least one inhibitor of the endogenous endothelin producing system, or
i2) in one separate container a pharmaceutical dosage form comprising a dually acting compound capable of inhibiting neutral endopeptidase and the endogenous endothelin producing system, and
ii) in another separate container a pharmaceutical dosage form comprising at least one PDE V inhibitor.

30. A kit according to claim 29, comprising in separate containers in a single package pharmaceutical dosage forms for use in combination, comprising:
i) in one separate container a pharmaceutical dosage form comprising a dually acting compound capable of inhibiting neutral endopeptidase and the endogenous endothelin producing system, and
ii) in another separate container a pharmaceutical dosage form comprising at least one PDE V inhibitor.
